(19) United States
(12) Patent Application Publication
(10) Pub. No.: US 2007/0122811 A1
(43) Pub. Date: May 31, 2007

(54) Compositions and processes for genotyping single nucleotide polymorphisms

(76) Inventor: Philip Buzby, Brockton, MA (US)

Correspondence Address: GIFFORD, KRASS, SPRINKLE, ANDERSON;& CITKOWSKI, P.C., P.O. BOX 7021, TROY, MI, 48007-7021, US

(21) Appl. No.: 10/574,551

(22) PCT Filed: Sep. 30, 2004
(86) PCT No.: PCT/US04/32164

§ 371 Date: Jul. 7, 2006

Related U.S. Application Data
(60) Provisional application No. 60/481,443, filed on Sep. 30, 2003.

(51) Int. Cl. C12Q 1/68; C12P 19/34; C12N 9/16

(52) U.S. Cl. 435/6; 435/91.2; 435/196

(57) ABSTRACT

The invention relates to processes, compositions and kits for analysis of nucleic acid variations, especially single nucleotide polymorphisms (SNPs). An inventive process includes a procedure to enzymatically remove inorganic pyrophosphate from a sample prior to and/or during a single base extension reaction. Processes and compositions described herein are especially useful in nucleic acid analysis methods designed to minimize transfer and separation steps.

### RELATED APPLICATION

This application claims priority of U.S. Provisional Patent Application Ser. No. 60/481,443 filed Sep. 30, 2003, which is incorporated herein by reference.

### FIELD OF THE INVENTION

The present invention relates to processes and compositions for detecting and characterizing a specified nucleotide in a nucleic acid sequence. Further, the invention relates to a processes and compositions for reducing misincorporation of a labeled nucleotide or nucleotide analog in a primer extension reaction.

### BACKGROUND OF THE INVENTION

DNA analysis is becoming increasingly important in the diagnosis of hereditary diseases, detection of infectious agents, tissue typing for histocompatibility, identification of individuals in forensic and paternity testing, and monitoring the genetic makeup of plants and animals in agricultural research (Alford, R. L., et al., Curr. Opin. Biotechnol. (1994) 5:29-33). In addition, DNA analysis is crucial in large-scale genetic studies to identify susceptibility alleles associated with common diseases involving multiple genetic and environmental factors (Risch, N., et al., Science (1996) 273:1516-1517). Recently, attention is focused on single nucleotide polymorphisms (SNPs), the most common DNA sequence variation found in mammalian genomes (Cooper, D. N., et al., Hum Genet (1985) 69:201-205). While most of the SNPs do not give rise to detectable phenotypes, a significant fraction of them are disease-causing mutations responsible for genetic diseases. As the DNA sequence of the human genome is completely elucidated, large-scale DNA analysis will play a crucial role in determining the relationship between genotype (DNA sequence) and phenotype (disease and health) (Cooper, D. N., et al., Hum Genet (1988) 78:299-312). Although some assays have considerable promise for high throughput, the recently developed DNA diagnostic processes all require specialty reagents and expensive detection instrumentation. Such processes include the high-density chip arrays for allele-specific hybridization analysis as described by Pease, A. C., et al., Proc Natl Acad Sci USA (1994) 91:5022-5026; Yershov, G., et al., Proc Natl Acad Sci USA (1996) 93:4913-4918, and Wang, D. G., et al., Science (1998) 280:1077-1081; the homogeneous 5′-nuclease allele-specific oligonucleotide cleavage assay TaqMan ASO detailed in Livak, K. J., et al., Nat Genet (1995) 9:341-342, and Whitcombe, D., et al., Clin Chem (1998) 44:918-923; a homogeneous fluorescence assay for PCR amplification: its application to real-time, single-tube genotyping, the homogeneous template-directed dye-terminator incorporation (TDI) assay detailed in Chen, X., et al., Nucleic Acids Res (1997) 25:347-353 and Chen, X., et al., PNAS USA (1997) 94:10756-1076; the homogeneous dye-labeled oligonucleotide ligation (DOL) assay described by Chen, X. et al. Genome Research (1998) 8: 549-556; and the homogeneous molecular beacon ASO assay of Tyagi, S. et al. Nature Biotechnology (1998) 16: 49-53.

A particularly important genotyping technique is template-directed primer extension—a chain terminating DNA process designed to ascertain the nature of the one base immediately 3′ to the sequencing primer that is annealed to the target DNA immediately upstream from the polymorphic site. In the presence of DNA polymerase and the appropriate terminator, e.g. a dideoxyribonucleoside triphosphate (ddNTP), the primer is extended specifically by one base as dictated by the target DNA sequence at the polymorphic site. By determining which terminator is incorporated, the allele(s) present in the target DNA can be inferred. This genotyping process has been widely used in many different formats and proven to be highly sensitive and specific as illustrated in Syvanen, A.-C et al, Genomics (1990) 8: 684-692 and Syvanen, A.-C. and Landegren, U. Human Mutation (1994) 3: 172-179. However, in some cases such genotyping processes are less robust than desired due to misincorporation of a signaling molecule, e.g. the wrong labeled terminator, leading to weak and ambiguous results. Such problems often require troubleshooting and modification of reaction conditions, obviating many of the advantages of automated technologies and adding to the cost of nucleic acid analysis. Thus, there is a continuing need for compositions and processes to make nucleic acid analysis, such as genotyping by single base extension, more reliable and cost effective.

### SUMMARY OF THE INVENTION

A process for inhibiting misincorporation of a terminator in a single base primer extension reaction includes the step of providing a product of a nucleic acid synthesis reaction which contains nucleic acid template and a quantity of inorganic pyrophosphate. The product is incubated with an inorganic pyrophosphatase so as to decrease the quantity of pyrophosphate, yielding a purified reaction product. A further step includes combining the purified reaction product, a primer, a terminator having a detectable label, and a polymerase to form a mixture. This mixture is incubated under conditions sufficient to extend the primer by addition of the terminator, in one embodiment an acyclo nucleoside terminator, in a single base primer extension reaction.

The nucleic acid synthesis product further includes a residual reaction component remaining from the nucleic acid synthesis reaction. For instance residual primers and nucleotides are generally present. An optional step includes adding an exonuclease, an alkaline phosphatase, or a combination of those to the nucleic acid synthesis product or the purified nucleic acid synthesis product and incubating the product and enzyme under conditions sufficient to degrade a residual reaction component.

Optionally included in a detailed inventive process is the step of inactivating the residual reaction component removing enzyme. Also optional is a step of inactivating the inorganic pyrophosphatase.

In one embodiment of a provided process, a detectable label is a fluorescent label, an isotopic moiety, a mass tag, a peptide moiety, a carbohydrate moiety or a combination of these.

Another step in an inventive process is that of detecting the detectable label such as by detection of fluorescence polarization, direct fluorescence detection, fluorescence quenching, fluorescence anisotropy, time resolved fluorescence and fluorescence energy transfer. Other optional detection steps include radiation detection, mass spectrometry, and chromophore detection.

Optionally, the alkaline phosphatase is selected from the group consisting of: bacterial alkaline phosphatase, calf intestinal alkaline phosphatase or a combination of these. A preferred alkaline phosphatase is shrimp alkaline phosphatase. In an additional option, the exonuclease is selected from among lambda exonuclease, mung bean exonuclease, Bal31 exonuclease, T7 exonuclease and a combination thereof. A preferred exonuclease is exonuclease I. Optionally, a combination of shrimp alkaline phosphatase and exonuclease I is used.

A polymerase included in an inventive process is optionally a thermostable polymerase having a greater affinity for a terminator, in one embodiment an acyclo nucleoside terminator, than for a dideoxyterminator.

In another option, the inorganic pyrophosphatase is selected from among a mammalian inorganic pyrophosphatase, a bacterial inorganic pyrophosphatase, a yeast inorganic pyrophosphatase, and a combination of these. Additionally, the inorganic pyrophosphatase may be a thermostable inorganic pyrophosphatase.

In a preferred option, the steps of an inventive process are performed in a single reaction container such as in a single tube, well, concavity or the like. In another preferred option, a nucleic acid template or primer is included in an array.

In a further embodiment of a provided process for inhibiting misincorporation of a terminator in a single base primer extension reaction, the step of incubating a nucleic acid synthesis product and a pyrophosphate removing enzyme is included. A pyrophosphate removing enzyme is selected from among a pentosyltransferase, a phosphotransferase, a nucleotidyl transferase and a carboxylase and reaction is performed under conditions sufficient to decrease the quantity of pyrophosphate thereby yielding a purified reaction product. The purified reaction product is combined with a primer, a terminator having a detectable label, and a polymerase to form a mixture which is incubated under conditions sufficient to extend the primer by addition of the acyclo nucleoside terminator in a single base primer extension reaction.

Further provided is a process according to the invention for inhibiting misincorporation of a terminator in a single base primer extension reaction which includes the step of combining a nucleic acid template, a primer, an inorganic pyrophosphatase, a terminator, and a polymerase to form a mixture substantially free of deoxynucleotide-triphosphates. In a preferred embodiment the terminator is an acyclo nucleoside terminator. Also included is the step of incubating the mixture under conditions sufficient to extend the primer by addition of the terminator, wherein the pyrophosphatase inhibits pyrophosphorolysis in the single base primer extension reaction, thereby reducing misincorporation of a terminator.

In a preferred option, the included polymerase has higher affinity for an acyclo nucleoside terminator than for a dideoxynucleotide terminator, such as an AcycloPol™. Also optionally, the polymerase is a thermostable polymerase.

In one embodiment, the primer includes a 3′ terminal nucleotide complementary to the interrogation site nucleotide. Further provided is a process in which the primer includes a nucleotide complementary to the interrogation site and wherein the nucleotide is 2-10 nucleotides upstream of the 3′ terminal nucleotide of the primer.

Preferably, the acyclo nucleoside terminator includes a detectable label, which is optionally a fluorescent label.

Also provided is a composition according to the invention including an inorganic pyrophosphatase; a residual component removal agent selected from among an alkaline phosphatase, an exonuclease, or a combination thereof. Also included is a carrier. Optionally, the ratio of enzyme activity units of residual component removal agent to enzyme activity units of inorganic pyrophosphatase ranges between 1000:1-1:1000. In a further option, the ratio of enzyme activity units of residual component removal agent to enzyme activity units of inorganic pyrophosphatase ranges between 100:1-1:100. In another option, the ratio of enzyme activity units of residual component removal agent to enzyme activity units of inorganic pyrophosphatase ranges between 10:1-1:10.

An inventive composition includes an alkaline phosphatase including bacterial alkaline phosphatase, calf intestinal alkaline phosphatase or a combination of these. Preferably, the alkaline phosphatase is shrimp alkaline phosphatase. In addition, an exonuclease included in a composition according to the invention is optionally selected from among lambda exonuclease, mung bean exonuclease, Bal31 exonuclease, T7 exonuclease and a combination of these. Preferably, the exonuclease is exonuclease I.

An additional composition provided by the present invention includes an acyclo nucleoside terminator; an inorganic pyrophosphate; a pyrophosphatase; and a carrier. Preferably, the acyclo nucleoside terminator comprises a detectable label.

A pyrophosphatase included in an inventive composition is optionally a yeast inorganic pyrophosphatase and further optionally is selected from among a bacterial inorganic pyrophosphatase, a mammalian inorganic pyrophosphatase. Combinations of pyrophosphatases may also be included.

A commercial package is provided by the present invention which includes a mixture of an exonuclease, an alkaline phosphatase, an inorganic pyrophosphatase, and a carrier; and instructions for use of the mixture in a primer extension reaction. In a preferred option, the exonuclease is exonuclease I. Also preferred is an embodiment in which the alkaline phosphatase is shrimp alkaline phosphatase. Further optionally, the pyrophosphatase is a yeast pyrophosphatase. A thermostable pyrophosphatase, a bacterial pyrophosphatase or a mammalian pyrophosphatase may also be included.

An inventive commercial package also optionally includes a mixture having an additive selected from among the group including a chelator, a polyol, a reducing agent, a protease inhibitor, a detergent, and a combination of these. Also included is an embodiment in which the carrier is a buffered solution.

An embodiment of the present invention provides use of an inorganic pyrophosphatase in a process for identification of an interrogation site by single base extension. Additionally provided is a process for determining the identity of a nucleotide at an interrogation site, a composition comprising an inorganic pyrophosphatase, and a commercial package comprising an inorganic pyrophosphatase, all essentially as described herein.

### BRIEF DESCRIPTION OF THE DRAWINGS

FIG. 1 is a drawing illustrating misincorporation of a terminator;

FIG. 2A is a drawing illustrating genotype analysis;

FIG. 2B is a drawing illustrating genotype analysis;

FIG. 2C is a drawing illustrating genotype analysis; and

FIG. 3 is a flow diagram illustrating an embodiment of an inventive method.

### DETAILED DESCRIPTION OF THE INVENTION

The present invention is directed towards processes and compositions which make detecting and characterizing a specified nucleotide in a nucleic acid sequence more reliable and cost effective. Particular applications of inventive processes and compositions include genotyping to identify a particular nucleotide in a nucleic acid sequence having a single nucleotide polymorphism (SNP). In this application, the term “interrogation site” as used herein is intended to mean a nucleotide in an oligonucleotide or polynucleotide whose identity is to be determined using an inventive process or composition.

A method of identifying a specified nucleotide in a nucleic acid sequence includes the steps of isolation and amplification of a nucleic acid template including an interrogation site, hybridization of the template with a primer, and extension of the primer by addition of a terminator, such that detection of incorporation of the terminator is indicative of the identity of the nucleotide at the interrogation site.

Misincorporation of a terminator in a process such as that described may result in erroneous identification of the nucleotide at the interrogation site. Pyrophosphate (PPi) is generated during polymerization of a nucleic acid and this molecule is implicated in the phenomenon of terminator misincorporation. In particular, misincorporation is demonstrated herein to be attributable to pyrophosphate stimulated pyrophosphorolysis, also termed reverse polymerization. The removal of a nucleotide from the primer may result in a site for addition of a terminator which would not be available in absence of such removal. For example, a terminator complementary to a nucleotide upstream of the interrogation site nucleotide may be added.

The term “complementary” as used herein is intended to indicate the identity of a nucleotide or nucleotide analog with reference to usual nucleic acid base pairing. For example, using the letter U, T, A, C and G to represent various nucleic acid bases, C is complementary to G and vice versa, A and T are complementary as are U and A. Referring to acyclo nucleoside terminators, AcyATP incorporated into a nucleic acid sequence is complementary to T, and so on.

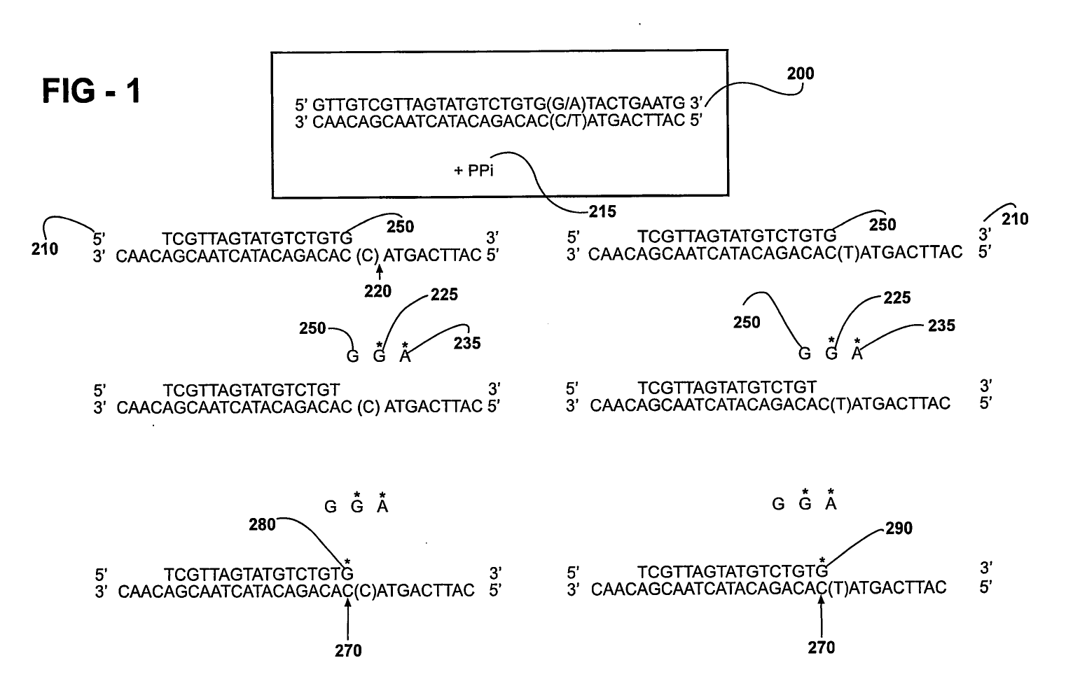

FIG. 1 illustrates an example of misincorporation of a labeled terminator in the presence of pyrophosphate. In this example, an assay to determine the identity of a nucleotide at an interrogation site in an amplified template 200 includes a SNP primer with a G at the 3′ end 210. The SNP primer 210 is hybridized to a complementary strand of the amplified DNA template including either a C at the interrogation site 220 or a T at the interrogation site 230. A DNA polymerase (not shown) is included to incorporate a labeled terminator 225 or 235 and thus extend the primer 210 by one base, the terminator complementary to the interrogation site 230. In the presence of pyrophosphate 215 generated during amplification such as PCR, the DNA polymerase will also catalyze the reverse reaction pyrophosphorolysis, and cleave the 3′ terminal base 250 from the primer 210. Although not wishing to be bound by theoretical considerations, it is believed that initially the forward reaction of polymerization is more efficient than the reverse polymerization pyrophosphorolysis reaction. However, once the forward reaction is completed and one of the dye terminators is substantially reduced in concentration, the reverse reaction becomes dominant. Depending on concentrations of terminators and pyrophosphate, one or more bases may be removed from the primer. Once one or more bases is removed from the primer by pyrophosphorolysis, the primer is extended by addition of a terminator 225 at a position 270 upstream of the interrogation site, a misincorporation. A misincorporation may be a “silent” misincorporation such as illustrated at 280 where a labeled terminator is incorporated at a site upstream of the interrogation site but is of the same type, here a G, that would be incorporated at the interrogation site. In contrast, a misincorporation such as shown at 290 is an error producing misincorporation since detection of the labeled terminator incorporated into the primer will be interpreted as though the nucleotide at the interrogation site is a C rather than a T.

Of particular interest is the fact that the primer shown at 210 includes a nucleotide 250 that can be replaced by a terminator such that there is correct hybridization between the terminator and the nucleotide at the corresponding position 270 in the amplified template strand. Such a primer 210 is identified by the present invention as a primer “susceptible to misincorporation,” discussed further below.

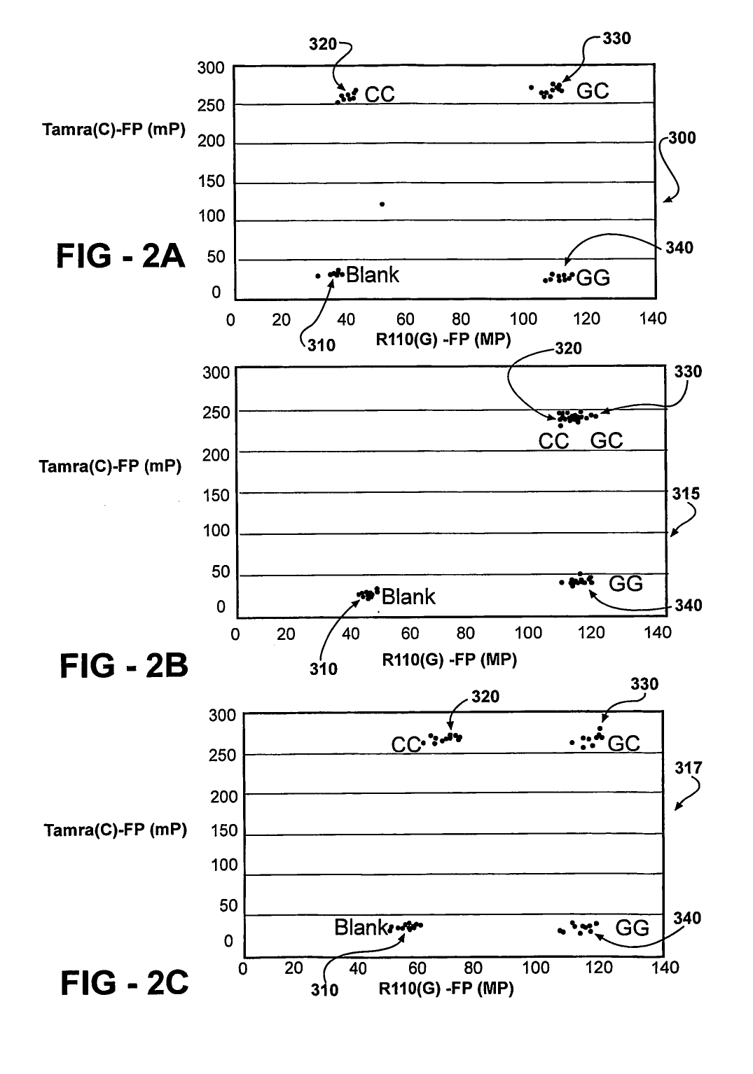

FIG. 2 illustrates the consequence of misincorporation on analysis of genotype as well as the effect of pyrophosphate removal in decreasing misincorporation. In this example, detected changes in fluorescence polarization indicate incorporation of a specified fluorescently labeled terminator into a primer. FIG. 2A shows a result 300 of primer extension analysis in which fluorescently labeled G or C terminators were incorporated in absence of pyrophosphate. Data point cluster 310 indicates a negative control in which no template is present for either the forward or reverse polymerization reaction to occur. Clusters 320, 330 and 340 indicate combinations of dye terminators CC, GC and GG, respectively, detected by a fluorescence detector. FIG. 2B shows a result 315 of analysis of a primer extension reaction including pyrophosphate which resulted in terminator misincorporation. In this example it can be seen that the data point cluster representing the CC genotype 320 is shifted right towards the GC data point cluster 330 as compared to the position of this cluster in FIG. 2A. This shift results in error in interpreting the genotype of the template DNA. FIG. 2C exemplifies a result in an assay performed as in 2B except that pyrophosphate is removed by incubation with pyrophosphatase.

Thus, in one embodiment of a process according to the present invention pyrophosphorolysis is inhibited in order to suppress misincorporation of a terminator in a nucleic acid synthesis reaction. In particular pyrophosphorolysis is suppressed by reduction of pyrophosphate concentrations in a nucleic acid synthesis reaction.

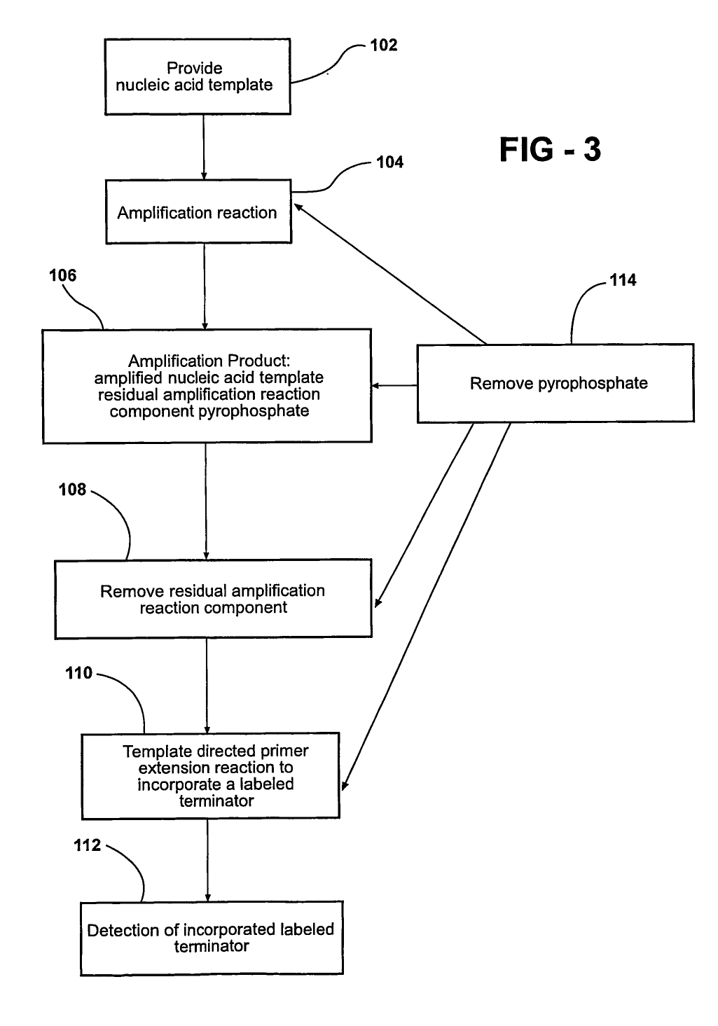

An embodiment of an inventive process is illustrated schematically in FIG. 3 including a step 102 of providing a nucleic acid template having an interrogation site, such as genomic DNA or DNA from reverse transcription of RNA. A nucleic acid template provided for use in an inventive process is isolated from any of various species, including a rodent, particularly rat or mouse; an avian, particularly chicken; a ruminant; a microbial species, particularly a bacterium; a plant; and a virus. In one embodiment of an inventive process, a provided nucleic acid template is isolated from a primate, particularly a human.

A nucleic acid template may be isolated according to standard methods such exemplified in Sambrook, J. et al., Eds. “Molecular Cloning, A Laboratory Manual”, Cold Spring Harbor Press, Cold Spring Harbor, N.Y. (1989) and in Ausubel, F. A. et al. Eds. “Current Protocols in Molecular Biology” John Wiley and Sons, New York, N.Y. (1994). Further, a nucleic acid template may isolated from any of various cells, tissues or bodily fluids, illustratively including those from animal sources such as blood, saliva, mucus, tears or urine. In addition, a nucleic acid template may be isolated from cells such as epidermal cells; epithelial cells; mucosal cells; hair roots; spermatozoa; and leukocytes; and from tissues or organs illustratively including blood vessel, bone, brain, digestive organ, endocrine or exocrine tissue, heart, kidney, liver, lung, lymph node, nasal epithelium, nerve, reproductive organ, spleen, spinal tissue.

Another step 104 included in an embodiment of an inventive method is amplification of the nucleic acid template. In a particular embodiment, an amplification step includes a PCR reaction. Other amplification methods may be used, such as strand displacement amplification described in U.S. Pat. Nos. 5,455,166; 5,648,211; 5,712,124; and 5,744,311, ligase chain reaction as disclosed in Eur. Pat. Appl. No. 320308, gap LCR such as described in Wolcott, M. J., Clin. Microbiol. Rev. 5:370-386, a NASBA technique as described in Guatelli J. C. et al. Proc. Natl. Acad. Sci. USA 87:1874-1878, 1990, an amplification as described in European Patent Application No. 4544610, and, target mediated amplification as described in PCT Publication WO 9322461.

An amplification product 106 results from the amplification reaction, the amplification product 106 including an amplified nucleic acid template, a residual amplification reaction component and inorganic pyrophosphate.

A residual amplification reaction component includes such components as an unincorporated nucleotide and an excess primer. Since such residual components interfere with primer extension, a further step 108 includes degradation of a primer and/or a free nucleotide in the amplification reaction product. For example, an exonuclease is used to degrade a single stranded nucleic acid primer in an amplification product. Exemplary exonucleases that may be used to degrade residual primers in an amplification reaction product include lambda exonuclease, mung bean exonuclease, Bal31 exonuclease and T7 exonuclease. In a particular embodiment the exonuclease is exonuclease I.

An alkaline phosphatase may be used in an inventive process to degrade a residual nucleotide such that the nucleotide is no longer capable of being incorporated into a nucleic acid chain, such as by catalyzing the hydrolysis of 5′-phosphate groups of a nucleotide. In a particular embodiment the alkaline phosphatase is shrimp alkaline phosphatase. Other suitable alkaline phosphatases illustratively include a bacterial alkaline phosphatase and a calf intestinal alkaline phosphatase.

Pyrophosphate (PPi) is generated during polymerization of a nucleic acid and pyrophosphate is therefore present in the amplification product 106. As described herein pyrophosphate is implicated in generation of error in an included primer extension step 110.

A step 114 included in an embodiment of an inventive process is removal of pyrophosphate from an amplification product.

Pyrophosphate levels are decreased by introduction of an enzyme that removes a pyrophosphate molecule. Such enzymes illustratively include a pentosyltransferase, a phosphotransferase, a nucleotidyl transferase and a carboxylase such as those described in U.S. Pat. No. 6,291,164.

In a particular embodiment the enzyme for removal of pyrophosphate is an inorganic pyrophosphatase, such as included in the class of enzymes described by IUBMB Enzyme Nomenclature EC 3.6.1.1. Inorganic pyrophosphatases from various species are suitable in an inventive method illustratively including those described in Baykov, A. A., et al., (1999) Progr. Mol. Subcell. Biol. 23, 127-150; Sivula, T., et al., (1999) FEBS Lett. 454, 75-80; such as bacterial inorganic pyrophosphatases: Young, T. W., et al., (1998) Microbiology 144, 2563-2571, Merckel, M. C., et al., (2001) Structure (London) 9, 289-297, Josse, J. (1966) J. Biol. Chem. 231, 1938-1947, Parfenyev, A. N., et al., (2001) J. Biol. Chem. 276, 24511-24518, Wang, S. C. K., et al., (1970) J. Biol. Chem. 245, 4335-4345; yeast inorganic pyrophosphatases: Baykov, A. A. and Avaeva, S. M. (1974) Regulation of yeast inorganic-pyrophosphatase activity by divalent cations. Eur. J. Biochem. 47, 57-66, Kolakowski, Jr, L. F., et al., (1988) Nucleic Acids Res. 16, 10441-10452; and mammalian inorganic pyrophosphatases: Smirnova, I. N., et al., (1988) Arch. Biochem. Biophys. 267, 280-284, Smirnova, I. N., et al., (1995) Arch. Biochem. Biophys. 318, 340-348. An inorganic pyrophosphatase may be isolated from an organism which naturally produces the protein by standard protein isolation techniques, such as described in Roe, S., Protein Purification Techniques: A Practical Approach, Oxford University Press; 2nd ed. (2001); or Scopes, R. K., Protein Purification: Principles and Practice, Springer-Verlag; 3rd ed. (1994). Alternatively, an inorganic pyrophosphatase is produced by cloning and expression of the protein in a native or heterologous organism from which it is isolated for use in an inventive method and composition. For example, cloned versions of an inorganic pyrophosphatase are described for bacterial, mammalian and yeast inorganic pyrophosphatases. Specific examples are described in Maruyama, S. et al., Biochem. Mol. Biol. Int., 40:679-688 (1996); Fairchild, T. A. and Patejunas, G., Biochim. Biophys. Acta, 1447:133-136 (1999); and Kolakowski, L. F. et al., Nucl. Acids Res., 16:10441-10452 (1988). Further, thermostable versions of inorganic pyrophosphatases, illustratively including a pyrophosphatase described in WO 02/088387 or WO 94/05797 are included in some embodiments of the present invention.

The step of removing pyrophosphate 114 includes decreasing pyrophosphate levels such that pyrophosphorolysis is inhibited and nucleic acid synthesis is favored. Following amplification, pyrophosphate is typically present in micromolar concentrations in an amplification reaction product. In an inventive process, treatment of an amplification reaction product with a pyrophosphate removal enzyme as described herein removes sufficient pyrophosphate such that DNA synthesis is favored as demonstrated by reduction in frequency of misincorporation. In an exemplary pyrophosphatase removal step, a solution of pyrophosphatase has a concentration of 0.2 units per microliter, and 0.1 to 1 microliter is incubated with a PCR product at 37° for times ranging from 1 minutes to 1 hour. A reduction in misincorporation events is observed following such treatment. One unit of pyrophosphatase is defined as degrading 1 micromole of pyrophosphate in 1 minute at 25° C.

Removal of pyrophosphate 114 from an amplification product is optionally combined with removal of a residual amplification reaction component 108. In one embodiment, removal of pyrophosphate is combined with removal of residual nucleotides in the amplification product, removal of residual primers in the amplification product, or a combination thereof. In another embodiment, a pyrophosphatase is provided in a mixture including an exonuclease and/or an alkaline phosphatase, as described below.

The amplified nucleic acid template is used in a template directed primer extension reaction 110. In one embodiment the template directed primer extension reaction 110 is a single base extension reaction which includes annealing a primer to an amplified nucleic acid template and adding a labeled terminator to the 3′ end of the primer. The added labeled terminator is complementary to an interrogation site in the template.

In general, a primer extension reaction 110 includes a primer to be extended, a template which directs the identity of the molecule added to the primer, a polymerase and at least one type of nucleotide, nucleotide-based nucleotide analog, or acyclo-based analog capable of being added to the terminus of a nucleic acid primer and further capable of specific base-pairing with a nucleotide present in a complementary nucleic acid. A primer extension reaction is performed in a suitable medium, generally an aqueous buffered medium, such as Tris-HCl. Appropriate ionic co-factors may be included, such as MgSO₄ and the like. Primer extension reactions illustratively include standard primer extension reactions such as those set forth in Sambrook, J. et al., Eds. “Molecular Cloning, A Laboratory Manual”, Cold Spring Harbor Press, Cold Spring Harbor, N.Y. (1989) and in Ausubel, F. A. et al. Eds. “Current Protocols in Molecular Biology” John Wiley and Sons, New York, N.Y. (1994).

In a preferred embodiment, the primer extension reaction is a single base primer extension reaction. A single base primer extension reaction includes a primer to be extended, a template which directs the identity of the molecule added to the primer, a polymerase and at least one type of terminator. As used herein, the term “terminator” or “chain terminating nucleotide” refers to a nucleotide, nucleotide-based nucleotide analog, or acyclo-based analog capable of being added to the terminus of a nucleic acid primer and further capable of specific base-pairing with a nucleotide present in a complementary nucleic acid and which prevents further chain elongation after incorporation at the terminus of a nucleic acid chain. Exemplary terminators include 2′,3′-dideoxynucleotides such as ddATP, ddGTP, ddCTP and ddTTP. Analogs of 2′,3′-dideoxynucleotide terminators are also included, for example, 5-bromo-dideoxyuridine, 5-methyl-dideoxycytidine and dideoxyinosine are suitable analogs. Other 3′-deoxynucleoside analogs may also be used as terminator nucleotides.

A particularly preferred terminator included in a primer extension according to the invention is an acyclo nucleoside terminator, such as described in U.S. Pat. Nos. 5,332,666 and 5,558,991.

A terminator may include a detectable label such as a fluorophore, an isotopic moiety or a mass tag. Also, a detectable protein or peptide such as an antigen, hapten, ligand, antibody, receptor, enzyme or substrate may be included as a detectable label.

Exemplary fluorophores include fluoresceins, such as fluorescein isothiocyanate (FITC), 6-carboxy-2′,4,4′,5′,7,7′-hexachlorofluorescein, 6-carboxy-4′,5′-dichloro-2′,7′-dimethoxyfluorescein; rhodamines, such as N,N,N′,N′-tetramethyl-6-carboxyrhodamine, 6-carboxy-X-rhodamine, 5-carboxyrhodamine-6G, 6-carboxyrhodamine-6G; cyanines, such as Cy3, Cy5 and Cy7; coumarins; phenanthridines, such as Texas Red; and the like. Fluorophores also include R110 and Tamra.

A particularly preferred terminator included in an embodiment of an inventive method and composition is a dye-labeled AcycloTerminator™ such as those commercially available from PerkinElmer®. An exemplary dye-labeled AcycloTemminator, AcyATP, is illustrated below:

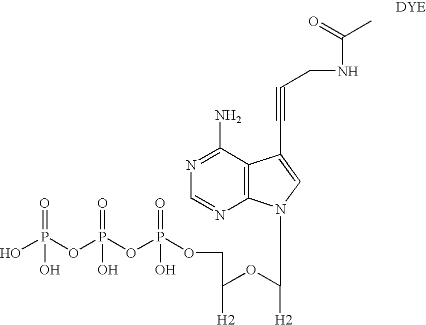

Other dye-labeled AcycloTerminators, including Acy UTP, AcyTTP, AcyGTP, and AcyCTP are also included in some embodiment of a process according to the invention.

In a particularly preferred embodiment a single base primer extension reaction includes a primer to be extended, a template which directs the identity of the molecule added to the primer, a dye-labeled acyclo nucleoside terminator such as a dye-labeled AcycloTerminator®, and a polymerase having a higher affinity for the terminator than a specified Taq DNA polymerase. In particular, an AcycloPol™ polymerase is included according to one embodiment of an inventive method. See for example, Gardner, A. F. and Jack, W. E. “Acyclic and dideoxy terminator preferences denote divergent sugar recognition by archaeon and Taq DNA polymerases”, Nucleic Acids Res. 30, 605-613. Examples of suitable polymerases are described further in U.S. Pat. Nos. 5,352,778; 5,500,363; and 5,756,334. Such polymerases and methods are further described in WO 0123411 “Incorporation of Modified Nucleotides By Archeon DNA Polymerases And Related Methods.”

A step 112 in a genotyping method is detection of the incorporated labeled nucleotide such that the identity of the labeled nucleotide is revealed. In one embodiment, the identity of the incorporated labeled nucleotide allows inference of the identity of the interrogation site since the labeled nucleotide is complementary to the interrogation site. Detection of a labeled nucleotide is performed by any of various methods depending on the type of label. For example, illustrative detection methods include direct fluorescence detection, fluorescence quenching, fluorescence anisotropy, time resolved fluorescence and fluorescence energy transfer. Exemplary methods are described in B. Valeur, “Molecular Fluorescence: Principles and Applications”, Wiley-VCH, 2001 and J. R. Lakowicz, “Principles of Fluorescence Spectroscopy”, Plenum Publishing, 1999. Other detection methods include radiation detection, such as by autoradiography and scintillation detection, mass spectrometry, and enzyme reaction product detection. A further detection method includes detection of fluorescence polarization such as described in Chen et al., Genome Res., 9:492-498, 1999 and in U.S. Pat. Nos. 6,180,408 and 6,440,707.

A single base extension reaction according to the invention may be performed in single tubes, multiwell microplates, microchannel devices, flat surfaces with or without depressions, upon microarrays, and the like. In a particular embodiment, the steps of an inventive process are performed in a single container by successive addition of reagents and incubation under appropriate conditions.

In another embodiment, a pyrophosphatase is added to remove pyrophosphate during a polymerization reaction in which a terminator is present. In a particularly preferred embodiment, a pyrophosphatase is added to a polymerization reaction including an acyclo nucleoside terminator. In one embodiment, a pyrophosphatase is added to a polymerization reaction including a dye-labeled acyclo nucleoside terminator.

In a further embodiment of a process according to the present invention a pyrophosphatase is included in an amplification reaction, such as a PCR reaction. Optionally, a pyrophosphatase is added in more than one step according to an inventive method. For example, a pyrophosphatase is added to an amplification reaction and to an extension reaction. In another example, a pyrophosphatase is incubated with an amplification reaction product following the amplification reaction, optionally in concert with incubation of the reaction product and a residual reaction component removal agent. Generally, a residual reaction component removal agent must be inactivated prior to performing a primer extension reaction, which may decrease the activity of the inorganic pyrophosphatase. Thus, in one option, an inorganic pyrophosphatase is added to an amplification product and to an extension reaction. In a further option, steps of removing a residual amplification reaction component and inactivating a residual amplification reaction agent are performed prior to the step of adding an inorganic pyrophosphatase. Optionally, the pyrophosphatase in an amplification product is inactivated prior to use of the template in a primer extension reaction.

The inventor has discovered that certain primer sequences are “susceptible” to misincorporation. A primer susceptible to misincorporation is a primer included in a single base primer extension reaction wherein the primer includes a nucleotide at a specified position in the primer which can be replaced by a terminator following a pyrophosphorolysis event in the reaction mix. The terminator is incorporated such that correct pairing between the terminator and a complementary nucleotide at an analogous specified position in the amplified template occurs when the primer is annealed to the template.

For example, referring again to FIG. 2, a susceptible primer is illustrated in at 210. Primer 210 includes a nucleotide 250 at the 3′ terminus of the primer 210 which is removed from the primer by pyrophosphorolysis. The terminal nucleotide removed is replaced by a terminator 260 included in the reaction mix such that correct pairing between the incorporated terminator and a complementary nucleotide at the analogous specified position in the amplified template occurs when the primer is annealed to the template.

A majority of misincorporation events occur using a susceptible primer wherein the nucleotide removed by pyrophosphorolysis and replaced by a terminator is the terminal nucleotide of the primer. However, misincorporation events are also observed where more than one nucleotide is removed by pyrophosphorolysis and the terminator replaces a nucleotide 2-10 bases upstream of the terminus of the primer. Thus, in one embodiment a primer susceptible to misincorporation of a terminator in a single base primer extension reaction includes a nucleotide which can be replaced by a terminator following a pyrophosphorolysis event in the reaction mix such that correct pairing between the incorporated terminator and a complementary nucleotide at an analogous specified position in the amplified template occurs when the primer is annealed to the template wherein the nucleotide is 1-10 bases from the primer terminus.

Table 1 illustrates primer sequences tested in a template directed dye terminator incorporation assay with fluorescence polarization detection (TDI-FP). Protocols for TDI-FP are described in U.S. Pat. Nos. 6,180,408 and 6,440,707 and in examples detailed herein. In this table the template including an interrogation site is listed as “HapMap Marker” referring to a site in a single nucleotide polymorphism database, see http://www.ncbi.nlm.nih.gov/SNP/. Each “HapMap Marker” is identified by a “code” which corresponds to a SNP site (dbSNP) as noted in Table 2 below. In these assays, combinations of two fluorescently labeled terminators are used as indicated in the column “Dye Combo.” The first terminator of each listed pair is labeled with the fluorescent dye R110 while the second terminator of the pair is labeled with the fluorescent dye Tamra. Each of the primers misincorporated a terminator having the fluorescent dye indicated in the column “Direction of Misincorporation.” In a majority of cases, the initial 3′ terminal nucleotide on the primer, shown in bold font, is identical to a base which would correctly hybridize with one of the two possible at the single nucleotide polymorphism interrogation site. Thus, phosphorolysis followed by incorporation of one of the two terminators gives erroneous results.

TABLE 1

| | | | | |
|---|---|---|---|---|
| HapMap | Dye | Direction of | | SEQ ID |
| Marker | Combo | Misincorporation | TDI Primer Sequence | No. |

| | | | | |
|---|---|---|---|---|
| H052 E6 | C/T | Tamra | CCAAGAGGATAACTGCGGTCA | 1 |
| E7 | ″ | Tamra | CCTGACCATCTTATGGCAATTCATAGTTA | 2 |
| G7 | ″ | Tamra | TTTCATACTGCAGCAGCAAGTTTAAT | 3 |
| H054 A4 | C/T | Tamra | GTCAAACAACAATCTTTTCCCTTAGAGTT | 4 |
| A6 | ″ | R110 | TGTGGCCACCACCTTGC | 5 |
| B10 | ″ | R110 | GGCCATCTAGTAGCTCCTAGGT | 6 |
| C6 | ″ | R110 | TGGTCCATTAATTTCAACAGTGACTC | 7 |
| C7 | ″ | Tamra | ATTATTCACATTAAGGTAGTATAATTCATTGTTTTCTG | 8 |
| C8 | ″ | Tamra | CCAGACATGTTCCAAGAATGC | 9 |
| E8 | ″ | R110 | TGATTTTTAGTCTCCCCTGGTTCC | 10 |
| F4 | ″ | Tamra | TCCAGAGGGTCTCAAAGCAAAT | 11 |
| F8 | ″ | Tamra | GGGCATCATTAGAAAGGAACAAAGT | 12 |
| H11 | ″ | Tamra | AGTGAGAGGGTTGTCAATTTTAGAGA | 13 |
| H12 | ″ | Tamra | GCTGCTGTGCAGAGGGTG | 14 |
| H056 B7 | C/T | Tamra | TTTATTCATCCATATGCCATGAATATAAGTGAA | 15 |
| F2 | ″ | Tamra | AAGTAAAAGCCTGAACACAAGAAGAAAT | 16 |
| F4 | ″ | Tamra | GAGGAGATCTAGAACTAGACATTGATAT | 17 |
| H058 B6 | G/C | R110 | GATGTGAGTTTCTTGGTGATCAGTG | 18 |
| C4 | G/T | R110 | GGGTAAGTACAATTCCTTCTCCCAG | 19 |
| C6 | G/T | R110 | GTTATAATTCATCTTAAAATAATACCCTTTAAGCACTTA | 20 |
| D3 | G/T | Tamra | CGTGGAAGACATGTCTCTACTGAT | 21 |
| H060 C12 | G/A | Tamra | TTTCATTCTCTGTTTCTTAAAGAAAAAAACAGTTA | 22 |
| E8 | ″ | R110 | TGGGAGGCTGAGATGGGA | 23 |
| F9 | ″ | Tamra | CCTGTTACCAGTTTAAGGGGCA | 24 |
| H062 A3 | G/A | Tamra | ACAGGCGTGAGCCACC | 25 |
| A4 | ″ | Tamra | GGAGTGAAAACAAGAAGGGAGGA | 26 |
| A6 | ″ | Tamra | GGCCATCCCTGGTCTTCTAAC | 27 |
| A9 | ″ | Tamra | GTACCAGAAGATAGGAAAAGAGGGAA | 28 |
| B5 | ″ | Tamra | CTCAGCTAGAGGGAGGAAGAAC | 29 |
| C3 | ″ | R110 | TCAGAGAATGCCAGAACAAACATTAG | 30 |
| C7 | ″ | R110 | CCATCAACTAGAACTCTATGTGATTATATCTAAAG | 31 |
| C8 | ″ | Tamra | TGAGGACTCTAATGAAAACACAGACAA | 32 |
| D2 | ″ | Tamra | GGATAGTGACTAACAAGCTATTTATGCTCA | 33 |
| D7 | ″ | Tamra | GCAGATCACCTGAGGTCAGAA | 34 |
| H1 | ″ | Tamra (mainly) | CCCCAGTTGAAAGTCGGTGA | 35 |
| H5 | ″ | Tamra (mainly) | GGAAAATGCATTATGAACACGAGAGTAAA | 36 |
| H6 | ″ | R110 | CCTGGCTGGTTTATCCTAGAAAGAG | 37 |
| H064 B2 | C/T | Tamra | GCAAAACCAGCAATAAAATATCTTACCTTT | 38 |
| C1 | ″ | Tamra | CATATTAATCTCTTCACAGTACACATTTAATGA | 39 |
| C3 | ″ | Tamra | CACTACCACAAATTATGCAGTCAAGT | 40 |
| C8 | ″ | Tamra | GGAGGTGGAGGCCTCAC | 41 |
| D5 | ″ | Tamra | GTTCTGGAGGCTACAAGTGTGAAAT | 42 |
| D12 | ″ | Tamra | GTCCAGGCTGGTCTCAAACT | 43 |
| E3 | ″ | Tamra | AGGTAAGGGCTGTGATTAAAGCATA | 44 |
| G5 | ″ | Tamra | GGAATGTGACAGATGCTGATTGTTC | 45 |
| G7 | ″ | Tamra | AAAGCAAGTTGTTCAAAGCCACA | 46 |
| H11 | ″ | Tamra | TGACTGTGTACCAGCACATTCTATG | 47 |
| H066 B8 | C/T | Tamra | CTGGTGTGAGATCAGGAAATGAGA | 48 |
| C7 | ″ | Tamra | CAAATTACTAAACTTTAGTGAGCCTCAGTTT | 49 |
| E2 | ″ | Tamra | CAGGCTAGGATAGAAATTGGGATCAT | 50 |
| E8 | ″ | Tamra | AATGGCAGCCTGGATAACTCAT | 51 |
| E9 | ″ | Tamra | TTGTCTTCTACAAGGCCTATAGCAAT | 52 |
| F7 | ″ | Tamra | TGAAAGAACAGCTTGCCTTCTCAT | 53 |
| G4 | ″ | Tamra | CTTCTGCTCTAGACACTGACTGTTT | 54 |
| H4 | ″ | Tamra | AATGCTGCATATATTTAAAGTATTTTCCTGAAATAAT | 55 |
| H068 A3 | A/T | R110 | CCTCCCAAAGTGCTGGGATT | 56 |
| C10 | A/T | R110 | CGGGCCCAAAACTGTTATTT | 57 |
| F9 | C/A | Tamra | CTTAAAGATGAATCCCCAAATAAAATTTCCAAA | 58 |
| F10 | C/A | Tamra | CAGGCGTGAGCCACCA | 59 |
| F12 | C/A | Tamra | AAAGAAAATTAAGTCTGACTACACTACAGC | 60 |
| G9 | C/A | Tamra | AGGACCACAATAGGCAAAAAAAAAAAAAA | 61 |
| G11 | C/A | Tamra | GGACCAGCCCCAAATGTCA | 62 |
| H8 | C/A | R110 | AGATGACAGAGGCTCCATAC | 63 |
| H11 | C/A | Tamra | GCTGTGAGTAAAATCCATCCTACCTA | 64 |

TABLE 2

| Code | dBSNP |
|---|---|
| H052E6 | rs1482552 |
| H052E7 | rs2189841 |
| H052G7 | rs1522279 |
| H054A4 | rs2691597 |
| H054A6 | rs2190434 |
| H054C6 | rs1981601 |
| H054C7 | rs160343 |
| H054C8 | rs1852612 |
| H054E8 | rs2040850 |
| H054F4 | rs307019 |
| H054H11 | rs1569256 |
| H054H12 | rs1182137 |
| H056F2 | rs1534734 |
| H056F4 | rs849763 |
| H058B6 | rs177477 |
| H058C4 | rs2177714 |
| H058C6 | rs2389635 |
| H058D3 | rs340393 |
| H060C12 | rs732575 |
| H060E8 | rs2935265 |
| H060F9 | rs3823832 |
| H062A3 | rs3935624 |
| H062A4 | rs740122 |
| H062A6 | rs218153 |
| H062A9 | rs731847 |
| H062B5 | rs1378008 |
| H062C3 | rs1618858 |
| H062C7 | rs3810897 |
| H062C8 | rs2355123 |
| H062D2 | rs3735397 |
| H062D7 | rs3113654 |
| H062H1 | rs2534596 |
| H062H5 | rs2329452 |
| H062H6 | rs2214935 |
| H064B2 | rs1540828 |
| H064C1 | rs1722280 |
| H064C3 | rs1035245 |
| H064D5 | rs1347801 |
| H064D12 | rs1014771 |
| H064E3 | rs698626 |
| H064G5 | rs1532084 |
| H064G7 | rs1962353 |
| H064H11 | rs1003291 |
| H066C7 | rs2190538 |
| H066E2 | rs953874 |
| H066E8 | rs2527521 |
| H066F7 | rs888046 |
| H066G4 | rs2192676 |
| H066H4 | rs1965950 |
| H068A3 | rs2190643 |
| H068C10 | rs2030673 |
| H068F10 | rs217385 |
| H068F12 | rs2428618 |
| H068G11 | rs296336 |
| H068H8 | rs4075894 |
| H068H11 | rs4081645 |

In one embodiment, an array of various single-stranded oligonucleotides of known sequence are attached to a substrate for genetic analysis of a sample. For instance, a sample containing a test nucleic acid is brought into contact with an oligonucleotide array under conditions which favor hybridization between the test nucleic acid sequence and complementary oligonucleotides present in the array. A DNA polymerase is added, along with a labeled terminator, such as an acyclo nucleoside terminator. Where hybridization between a test sequence and an oligonucleotide occurs, the DNA polymerase extends the oligonucleotide by a single base. Detection of a particular pattern of terminator incorporation within the array allows determination of allele and/or sequence information in the test nucleic acid. A pyrophosphatase is included in a method of arrayed primer extension to inhibit misincorporation of a terminator. In a particular embodiment, a pyrophosphatase is incubated with a test nucleic acid to reduce a quantity of pyrophosphate present. In a further embodiment, a pyrophosphatase is included in an arrayed primer extension reaction.

In a further embodiment of an inventive process, a primer included in a single base primer extension reaction has an initial 3′ terminus which extends to the interrogation site rather than ending one base upstream. In this embodiment, hybridization conditions are optimized such that addition of a terminator to the initial 3′ terminus of the primer will not occur unless the initial 3′ terminus is complementary to the interrogation site. Thus, under these conditions, following hybridization of the primer to the template, addition of a terminator to the initial 3′ terminus is indicative of correct hybridization of the initial 3′ terminus to the interrogation site and is therefore indicative of the identity of the nucleotide at the interrogation site. A step in a process according to such an embodiment of the invention is incubation of the template with a pyrophosphatase to remove a quantity of pyrophosphate.

Compositions

A composition for use in reducing misincorporation of a terminator in a primer extension reaction is provided by the present invention including a pyrophosphate reducing enzyme. Pyrophosphate reducing enzymes such as a pentosyltransferase, a phosphotransferase, a nucleotidyl transferase and a carboxylase such as those described in U.S. Pat. No. 6,291,164 may be included. A particularly preferred enzyme for removal of pyrophosphate included in an embodiment of an inventive composition is an inorganic pyrophosphatase such as described herein. A suitable inorganic pyrophosphatase is selected from any of various organisms including a microbial organism such as a bacterial or yeast inorganic pyrophosphatase.

In one embodiment, a pyrophosphate reducing enzyme is provided in a mixture with a component selected from the group consisting of: an exonuclease, an alkaline phosphatase. Exemplary exonucleases included in an inventive composition include lambda exonuclease, mung bean exonuclease, Bal31 exonuclease and T7 exonuclease. In a preferred embodiment the exonuclease includes exonuclease I.

An alkaline phosphatase may be included in an inventive composition. In a preferred embodiment an included alkaline phosphatase is shrimp alkaline phosphatase. Other suitable alkaline phosphatases illustratively include a bacterial alkaline phosphatase and a calf intestinal alkaline phosphatase.

In a further embodiment of an inventive composition, a carrier is included in the composition. In general, the carrier is in liquid or gel form and in one embodiment a carrier is an aqueous carrier, including 0.1-99% water. An aqueous carrier may include a component illustratively including, a buffering agent, a stability enhancing component such as a protein and/or glycoprotein illustratively including albumin and the like. In addition, a carrier and/or a composition may be sterilized in order to inhibit bacterial growth, such as by filtration. A bacteriocidal or bacteriostatic agent is also optionally included. For example, a carrier may be a reaction, dilution or storage buffer.

Optionally, the composition further includes an additive illustratively including a chelator such as EDTA and EGTA; a polyol, such as glycerol, sucrose and trehalose; a reducing agent, such as dithiothreitol, dithioerythritol and β-mercaptoethanol, a protease inhibitor, a detergent, and a combination thereof.

In an embodiment of an inventive composition, the ratio of enzyme activity units of residual component removal agent to enzyme activity units of inorganic pyrophosphatase ranges between 1000:1-1:1000, inclusive. In another embodiment, the ratio of enzyme activity units of residual component removal agent to enzyme activity units of inorganic pyrophosphatase ranges between 100:1-1:100, inclusive. In a further embodiment, the ratio of enzyme activity units of residual component removal agent to enzyme activity units of inorganic pyrophosphatase ranges between 10:1-1:10, inclusive.

In another embodiment, a composition for use in reducing misincorporation of a terminator in a single base extension reaction includes a primer susceptible to misincorporation, an inorganic pyrophosphate, and a pyrophosphatase. A carrier is also preferably included.

In a further embodiment of an inventive composition, a pyrophosphate removal enzyme is included in a primer extension composition. In particular, an inventive composition includes an acyclo nucleoside terminator and a pyrophosphate removal enzyme, particularly an inorganic pyrophosphatase as detailed herein.

Commercial Package

In one embodiment, a commercial package according to the invention includes a mixture of an exonuclease, an alkaline phosphatase, an inorganic pyrophosphatase, and an aqueous carrier. In addition, instructions for use of the mixture in a primer extension reaction are included in the commercial package.

In one option, an exonuclease included in an inventive composition is selected from lambda exonuclease, mung bean exonuclease, Bal31 exonuclease and T7 exonuclease and a combination thereof. In a further option, the preferred exonuclease includes exonuclease I.

Preferably, the alkaline phosphatase includes shrimp alkaline phosphatase. In a further option, the alkaline phosphatase is selected from the group including a bacterial alkaline phosphatase, a calf intestinal alkaline phosphatase and a combination thereof.

In one embodiment, the pyrophosphatase is a yeast pyrophosphatase, a bacterial pyrophosphatase, a mammalian pyrophosphatase or a combination of these. Optionally, the inorganic pyrophosphatase is a thermostable pyrophosphatase.

A carrier may be included in the mixture. In one embodiment a carrier is an aqueous carrier. In general, the carrier is in liquid or gel form and in one embodiment a carrier is an aqueous carrier, including 0.1-99% water. An aqueous carrier may include a component illustratively including, a buffering agent, a stability enhancing component such as a protein and/or glycoprotein illustratively including albumin and the like. In addition, a carrier and/or compositions may be sterilized in order to inhibit bacterial growth.

Optionally, the mixture further includes an additive illustratively including a chelator such as EDTA and EGTA; a polyol, such as glycerol, sucrose and trehalose; a reducing agent, such as dithiothreitol, dithioerythritol and β-mercaptoethanol, a protease inhibitor, a detergent, and a combination thereof.

In another embodiment, a commercial package includes a reagent for a process according to the invention, including an acyclo nucleoside terminator, a polymerase, a primer, or a combination of these. In one embodiment, a commercial package includes a fluorescent dye-labeled acyclo nucleoside terminator, a polymerase having a higher affinity for an acyclo nucleoside terminator than for a dideoxynucleotide terminator, a primer, or a combination of these.

It is appreciated that an enzyme for use in an inventive method may be supplied in a concentrated solution, which is generally more conducive to maintaining stability of a protein. Thus, a dilution buffer may be used in a method according to the invention and is optionally supplied in a commercial package.

Illustrative compositions and processes are presented in the following examples:

### EXAMPLE 1

Anonymous DNA samples of 96 individuals from the National Institutes of Health (NIH) Polymorphism Discovery Panel and CEPH family and publicly available markers from dbSNP database are used in this study. All primers are designed as described previously (Vieux et al. 2002) and are obtained from IDT. All reactions are run and read in 96-well or 384-well black plates from LabSource. Liquid handling instrument Evolution P3 (PerkinElmer) is used for assay assembly. PlatinumTaq DNA polymerase is from Invitrogen. AcycloPrime-FP SNP Kits are from PerkinElmer, including 10× reaction buffer, AcycloPol Enzyme for single base extension, PCR Clean-Up reagent (exonuclease I and shrimp alkaline phosphatase) and dilution buffer, and dye-labeled AcycloTerminator™ mixture, which contains equal amounts of R110 and TAMRA terminators. PPi is purchased from Sigma. Pyrophosphatase is from Roche. Texas red-labeled AcycloTerminators™ are from PerkinElmer. The assays are read in an Envison or Victor2 microplate reader (PerkinElmer) when R110 and TAMRA AcycloTerminators™ are used. For the combination of Texas red and TAMRA AcycloTerminators™, the assays are read in an Analyst microplate reader (Molecular Devices).

### EXAMPLE 2

Primer Extension Reaction Using Synthetic Oligodeoxynucleotides

For each primer extension reaction using synthetic oligodeoxynucleotides, two template oligodeoxynucleotides are synthesized, each with one of the two allelic bases at the target site. The heterozygous templates are made by mixing equal amounts of the two synthetic templates. The template sequences of four such reactions are summarized in Table 3.

TABLE 3
Sequence of Synthetic Oligodeoxynucleotides and Extension Primers

| Synthetic oligodeoxynucleotides | | |
|---|---|---|
| as extension templates | Extension primers | Misincorporation |
| Template 1 (G/C) | | |
| GTGGTGTTCCTCTTCGAAGGGCTTGCTAATCCTTGG | CCAAGGATTAGCAAGCCCTTCGAAGAG | G allele for CC |
| CCCA | | cluster |
| GTGGTGTTGCTCTTCGAAGGGCTTGCTAATCCTTGG | | |
| CCCA | | |
| Template 2 (G/A) | | |
| AGTGGATCCCACTGTCGATGGAGATGCCTGAGAAA | CTCAGGCATCTCCATCGACAGTGGG | G allele for AA |
| GACCC | | cluster |
| AGTGGACCCCACTGTCGATGGAGATGCCTGAGAAA | CTCAGGCATCTCCATCGACAGTGGG | |
| GACCC | | |
| Template 3 (A/T) | | |
| ACGAAAATTTTGCTAGTGGTTTACCCATGGTCATA | CTATGACCATGGGTAAACCACTAGCAAAA | A allele for TT |
| GCTGT | | cluster |
| ACGAAATTTTTGCTAGTGGTTTACCCATGGTCATAG | | |
| CTGT | | |
| Template 4 (G/A) | | |
| CAAAACTAAGATAGCTATTCCATCCCTCCATTCGTT | AACGAATGGAGGGATGGAATA | A allele for GG |
| TTTATTATC | | cluster |
| CAAAACTAAGATAGTTATTCCATCCCTCCATTCGTT | | |
| TTTATTATC | | |
| Template 5 (A/T) | | |
| CACTAGCAAAAATTTCGTGTCCTGTTCCCACTGGCC | GGCCAGTGGGAACAGGACACGAAA | A allele for TT |
| GTCG | | cluster |
| CACTAGCAAAATTTTCGTGTCCTGTTCCCACTGGCC | | |
| GTCG | | |
| Template 6 (G/A) | | |
| AGTGGATCCCACTGTCGATGGAGATGCCTGAGAAA | CTCAGGCATCTCCATCGACAGTGGG | G allele for AA |
| GACCC | | cluster |
| AGTGGACCCCACTGTCGATGGAGATGCCTGAGAAA | | |
| GACCC | | |

Forty-eight samples are prepared for each primer extension reaction, and each genotype has 12 samples; 100 nM synthetic templates are prepared in PCR buffer with or without PPi (100 microM). Seven microliters of synthetic template with or without PPi and 13 microliters TDI cocktail (containing 2 microliters 10 TDI reaction buffer, 0.5 microliters SNP primer [final concentration 384 nM], 0.05 microliters Acyclo-Pol enzyme, 1 microliters dye AcycloTerminator™ Mix, and 8.95 microliters water) are mixed and incubated from 2 min at 95° C., up to 70 cycles of single base extension for 15 sec at 95° C. and 30 sec at 55° C. After every five cycles, the product mixtures are read on an Envision fluorescence plate reader (PerkinElmer). To a mixture containing PPi and synthetic templates, 2 microliters Exo-SAP/pyrophosphatase mixture (1.8 microliters Exo-Sap buffer and 0.15 microliters pyrophosphatase) is added, and the reaction mixture is incubated for 1 h at 37° C. TDI cocktail (13 microliters) is then added, and the extension reaction is performed as above.

### EXAMPLE 3

PCR/TDI Reaction with or without Pyrophosphatase

All reactions are performed according to the manufacturer's manual. Briefly, DNA (3 ng) is amplified in 5 microliter reaction mixtures containing PCR primers and PCR reagents by using the following thermal cycling protocol: The mixture is held at 95° C. for 2 min followed by 40 cycles of 10 sec at 92° C., 20 sec at 58° C., and 30 sec at 68° C. The reaction mixtures are then incubated for 10 min at 68° C. before they are held at 4° C. until further use. Pyrophosphatase (1.5 microliters) is added to a stock solution of PCR clean-up enzyme mixture (10.5 microliters of 10× buffer, 1.33 microliters Exo-SAP). The PCR clean-up mixture (2 microliters) is added to 5 microliters PCR product mixture and incubated from 1 h at 37° C. to degrade the excess PCR primer, excess dNTP, and PPi generated during PCR. The enzymes are heat-inactivated for 15 min at 80° C. prior to the TDI reaction. TDI cocktail (13 microliters) is added to the reaction mixtures (7 microliters) from previous step. The reaction mixture is incubated for 2 min at 95° C., five to 70 cycles of 15 sec at 95° C. and 30 sec at 55° C. The final product mixtures are read on a fluorescence plate reader (PerkinElmer).

### EXAMPLE 4

An amplification product is produced containing approximately 2 micromoles pyrophosphate. A commercially available mixture of shrimp alkaline phosphatase and exonuclease I—Exo-SAP-IT® is combined with 0.03 units of yeast inorganic pyrophosphatase in a ratio of 1.33:1 in the amplification product. The mixture is incubated for 45 minutes, clearing 1.35 micromoles pyrophosphate.

### EXAMPLE 5

In one example of a reaction including a residual component reduction agent and a pyrophosphatase, 3 units of exonuclease I , 0.3 units of SAP and 10 mU of PPase added in 2 microliters to the final 5 microliters reaction. These are supplied as a 10× solution and diluted to IX before use with the PCR Clean-Up Dilution Buffer.

Further examples and details of inventive methods and compositions are described in Xiao et al., Role of Excess Inorganic Pyrophosphate in Primer-Extension Genotyping Assays., Genome Research 14:1749-1755 (2004).

This application claims priority of U.S. Provisional Patent Application Ser. No. 60/481,443 filed Sep. 30, 2003, which is incorporated herein by reference.

Any patents or publications mentioned in this specification are herein incorporated by reference to the same extent as if each individual publication was specifically and individually indicated to be incorporated by reference.

One skilled in the art will readily appreciate that the present invention is well adapted to carry out the objects and obtain the ends and advantages mentioned, as well as those inherent therein. The apparatus and processes described herein are presently representative of particular embodiments, exemplary, and not intended as limitations on the scope of the invention. Changes therein and other uses will occur to those skilled in the art. Such changes and other uses can be made without departing from the scope of the invention as set forth in the claims.

1. A process for inhibiting misincorporation of a terminator in a single base primer extension reaction, comprising:
providing a product of a nucleic acid synthesis reaction, the product comprising a nucleic acid template and a quantity of inorganic pyrophosphate;
incubating the product and an inorganic pyrophosphatase under conditions sufficient to decrease the quantity of pyrophosphate, to yield a purified reaction product;
combining the purified reaction product, a primer, a terminator having a detectable label, and a polymerase to form a mixture; and
incubating the mixture under conditions sufficient to extend the primer by addition of the terminator in a single base primer extension reaction, wherein decreasing the quantity of inorganic pyrophosphate in the product of a nucleic acid synthesis reaction inhibits pyrophosphorolysis in the single base primer extension reaction, so as to inhibit misincorporation of a terminator.

2. The process of claim 1 wherein the nucleic acid synthesis product further comprises a residual reaction component selected from the group consisting of: a residual primer and a nucleotide.

3. The process of claim 2 further comprising:
adding an enzyme selected from the group consisting of: an exonuclease, an alkaline phosphatase, and a combination thereof to the nucleic acid synthesis product; and
incubating the nucleic acid synthesis product and enzyme under conditions sufficient to degrade the residual reaction component.

4. The process of claim 2 further comprising:
adding an enzyme selected from the group consisting of: an exonuclease, an alkaline phosphatase, and a combination thereof to the purified reaction product; and
incubating the nucleic acid synthesis product and enzyme under conditions sufficient to degrade the residual reaction component.

5. The process of claim 3 further comprising:
inactivating the enzyme.

6. The process of claim 1 further comprising inactivating the inorganic pyrophosphatase.

7. The process of claim 1 wherein the detectable label is a fluorescent label.

8. (canceled)

9. The process of claim 1 further comprising detecting the detectable label.

10. The process of 9 wherein the step of detecting the label comprises detection of fluorescence polarization.

11-12. (canceled)

13. The process of claim 3 wherein the alkaline phosphatase is selected from the group consisting of: bacterial alkaline phosphatase, calf intestinal alkaline phosphatase and a combination thereof.

14. The process of claim 3 wherein the alkaline phosphatase is shrimp alkaline phosphatase.

15. The process of claim 3 wherein the exonuclease is selected from the group consisting of: lambda exonuclease, mung bean exonuclease, Bal31 exonuclease, T7 exonuclease and a combination thereof.

16. The process of claim 3 wherein the exonuclease is exonuclease I.

17. (canceled)

18. The process of claim 1 wherein the polymerase is a thermostable polymerase having a greater affinity for an acyclo nucleoside terminator than for a dideoxyterminator.

19-20. (canceled)

21. The process of claim 1 wherein the steps are performed in a single reaction container.

22. (canceled)

23. The process of claim 1 wherein the terminator is an acyclo nucleoside terminator.

24. The process of claim 23 wherein the acyclo nucleoside terminator comprises a detectable label.

25. (canceled)

26. A process for inhibiting misincorporation of a terminator in a single base primer extension reaction, comprising:
providing a product of a nucleic acid synthesis reaction, the product comprising a nucleic acid template and a quantity of inorganic pyrophosphate;
incubating the product and a pyrophosphate removing enzyme under conditions sufficient to decrease the quantity of pyrophosphate, to yield a purified reaction product;
combining the purified reaction product, a primer, a terminator having a detectable label, and a polymerase to form a mixture; and
incubating the mixture under conditions sufficient to extend the primer by addition of the terminator in a single base primer extension reaction, wherein decreasing the quantity of inorganic pyrophosphate in the product of a nucleic acid synthesis reaction inhibits pyrophosphorolysis in the single base primer extension reaction, so as to inhibit misincorporation of a terminator.

27. The process of claim 26 wherein the nucleic acid synthesis product further comprises a residual reaction component selected from the group consisting of: a residual primer and a nucleotide.

28. The process of claim 27 further comprising:
adding an enzyme selected from the group consisting of: an exonuclease, an alkaline phosphatase, and a combination thereof to the nucleic acid synthesis product; and
incubating the nucleic acid synthesis product and enzyme under conditions sufficient to degrade the residual reaction component.

29. The process of claim 27 further comprising:
adding an enzyme selected from the group consisting of: an exonuclease, an alkaline phosphatase, and a combination thereof to the purified reaction product; and
incubating the nucleic acid synthesis product and enzyme under conditions sufficient to degrade the residual reaction component.

30. The process of claim 26 further comprising inactivating the inorganic pyrophosphatase.

31. (canceled)

32. A process for inhibiting misincorporation of a terminator in a single base primer extension reaction, comprising:
combining a nucleic acid template, a primer, an inorganic pyrophosphatase, an acyclo nucleoside terminator, and a polymerase to yield a mixture substantially free of deoxynucleotide-triphosphates; and
incubating the mixture under conditions sufficient to extend the primer by addition of the acyclo nucleoside terminator, wherein the pyrophosphatase inhibits pyrophosphorolysis in the single base primer extension reaction, thereby reducing misincorporation of a terminator.

33-63. (canceled)


SEQUENCE LISTING (64 sequences)

SEQ ID NO: 1 - DNA, 21 bp, Artificial
  Synthetic Construct
ccaagaggataactgcggtca

SEQ ID NO: 2 - DNA, 29 bp, Artificial
  Synthetic Construct
cctgaccatcttatggcaattcatagtta

SEQ ID NO: 3 - DNA, 26 bp, Artificial
  Synthetic Construct
tttcatactgcagcagcaagtttaat

SEQ ID NO: 4 - DNA, 29 bp, Artificial
  Synthetic Construct
gtcaaacaacaatcttttcccttagagtt

SEQ ID NO: 5 - DNA, 17 bp, Artificial
  Synthetic Construct
tgtggccaccaccttgc

SEQ ID NO: 6 - DNA, 22 bp, Artificial
  Synthetic Construct
ggccatctagtagctcctaggt

SEQ ID NO: 7 - DNA, 26 bp, Artificial
  Synthetic Construct
tggtccattaatttcaacagtgactc

SEQ ID NO: 8 - DNA, 38 bp, Artificial
  Synthetic Construct
attattcacattaaggtagtataattcattgttttctg

SEQ ID NO: 9 - DNA, 21 bp, Artificial
  Synthetic Construct
ccagacatgttccaagaatgc

SEQ ID NO: 10 - DNA, 24 bp, Artificial
  Synthetic Construct
tgatttttagtctcccctggttcc

SEQ ID NO: 11 - DNA, 22 bp, Artificial
  Synthetic Construct
tccagagggtctcaaagcaaat

SEQ ID NO: 12 - DNA, 25 bp, Artificial
  Synthetic Construct
gggcatcattagaaaggaacaaagt

SEQ ID NO: 13 - DNA, 26 bp, Artificial
  Synthetic Construct
agtgagagggttgtcaattttagaga

SEQ ID NO: 14 - DNA, 18 bp, Artificial
  Synthetic Construct
gctgctgtgcagagggtg

SEQ ID NO: 15 - DNA, 33 bp, Artificial
  Synthetic Construct
tttattcatccatatgccatgaatataagtgaa

SEQ ID NO: 16 - DNA, 28 bp, Artificial
  Synthetic Construct
aagtaaaagcctgaacacaagaagaaat

SEQ ID NO: 17 - DNA, 28 bp, Artificial
  Synthetic Construct
gaggagatctagaactagacattgatat

SEQ ID NO: 18 - DNA, 25 bp, Artificial
  Synthetic Construct
gatgtgagtttcttggtgatcagtg

SEQ ID NO: 19 - DNA, 25 bp, Artificial
  Synthetic Construct
gggtaagtacaattccttctcccag

SEQ ID NO: 20 - DNA, 39 bp, Artificial
  Synthetic Construct
gttataattcatcttaaaataataccctttaagcactta

SEQ ID NO: 21 - DNA, 24 bp, Artificial
  Synthetic Construct
cgtggaagacatgtctctactgat

SEQ ID NO: 22 - DNA, 35 bp, Artificial
  Synthetic Construct
tttcattctctgtttcttaaagaaaaaaacagtta

SEQ ID NO: 23 - DNA, 18 bp, Artificial
  Synthetic Construct
tgggaggctgagatggga

SEQ ID NO: 24 - DNA, 22 bp, Artificial
  Synthetic Construct
cctgttaccagtttaaggggca

SEQ ID NO: 25 - DNA, 16 bp, Artificial
  Synthetic Construct
acaggcgtgagccacc

SEQ ID NO: 26 - DNA, 23 bp, Artificial
  Synthetic Construct
ggagtgaaaacaagaagggagga

SEQ ID NO: 27 - DNA, 21 bp, Artificial
  Synthetic Construct
ggccatccctggtcttctaac

SEQ ID NO: 28 - DNA, 26 bp, Artificial
  Synthetic Construct
gtaccagaagataggaaaagagggaa

SEQ ID NO: 29 - DNA, 22 bp, Artificial
  Synthetic Construct
ctcagctagagggaggaagaac

SEQ ID NO: 30 - DNA, 26 bp, Artificial
  Synthetic Construct
tcagagaatgccagaacaaacattag

SEQ ID NO: 31 - DNA, 35 bp, Artificial
  Synthetic Construct
ccatcaactagaactctatgtgattatatctaaag

SEQ ID NO: 32 - DNA, 27 bp, Artificial
  Synthetic Construct
tgaggactctaatgaaaacacagacaa

SEQ ID NO: 33 - DNA, 30 bp, Artificial
  Synthetic Construct
ggatagtgactaacaagctatttatgctca

SEQ ID NO: 34 - DNA, 21 bp, Artificial
  Synthetic Construct
gcagatcacctgaggtcagaa

SEQ ID NO: 35 - DNA, 20 bp, Artificial
  Synthetic Construct
ccccagttgaaagtcggtga

SEQ ID NO: 36 - DNA, 29 bp, Artificial
  Synthetic Construct
ggaaaatgcattatgaacacgagagtaaa

SEQ ID NO: 37 - DNA, 25 bp, Artificial
  Synthetic Construct
cctggctggtttatcctagaaagag

SEQ ID NO: 38 - DNA, 30 bp, Artificial
  Synthetic Construct
gcaaaaccagcaataaaatatcttaccttt

SEQ ID NO: 39 - DNA, 33 bp, Artificial
  Synthetic Construct
catattaatctcttcacagtacacatttaatga

SEQ ID NO: 40 - DNA, 26 bp, Artificial
  Synthetic Construct
cactaccacaaattatgcagtcaagt

SEQ ID NO: 41 - DNA, 17 bp, Artificial
  Synthetic Construct
ggaggtggaggcctcac

SEQ ID NO: 42 - DNA, 25 bp, Artificial
  Synthetic Construct
gttctggaggctacaagtctgaaat

SEQ ID NO: 43 - DNA, 20 bp, Artificial
  Synthetic Construct
gtccaggctggtctcaaact

SEQ ID NO: 44 - DNA, 25 bp, Artificial
  Synthetic Construct
aggtaagggctgtgattaaagcata

SEQ ID NO: 45 - DNA, 25 bp, Artificial
  Synthetic Construct
ggaatgtgacagatgctgattgttc

SEQ ID NO: 46 - DNA, 23 bp, Artificial
  Synthetic Construct
aaagcaagttgttcaaagccaca

SEQ ID NO: 47 - DNA, 25 bp, Artificial
  Synthetic Construct
tgactgtgtaccagcacattctatg

SEQ ID NO: 48 - DNA, 24 bp, Artificial
  Synthetic Construct
ctggtgtgagatcaggaaatgaga

SEQ ID NO: 49 - DNA, 31 bp, Artificial
  Synthetic Construct
caaattactaaactttagtgagcctcagttt

SEQ ID NO: 50 - DNA, 26 bp, Artificial
  Synthetic Construct
caggctaggatagaaattgggatcat

SEQ ID NO: 51 - DNA, 22 bp, Artificial
  Synthetic Construct
aatggcagcctggataactcat

SEQ ID NO: 52 - DNA, 26 bp, Artificial
  Synthetic Construct
ttgtcttctacaaggcctatagcaat

SEQ ID NO: 53 - DNA, 24 bp, Artificial
  Synthetic Construct
tgaaagaacagcttgccttctcat

SEQ ID NO: 54 - DNA, 25 bp, Artificial
  Synthetic Construct
cttctgctctagacactgactgttt

SEQ ID NO: 55 - DNA, 37 bp, Artificial
  Synthetic Construct
aatgctgcatatatttaaagtattttcctgaaataat

SEQ ID NO: 56 - DNA, 20 bp, Artificial
  Synthetic Construct
cctcccaaagtgctgggatt

SEQ ID NO: 57 - DNA, 20 bp, Artificial
  Synthetic Construct
cgggcccaaaactgttattt

SEQ ID NO: 58 - DNA, 33 bp, Artificial
  Synthetic Construct
cttaaagatgaatccccaaataaaatttccaaa

SEQ ID NO: 59 - DNA, 16 bp, Artificial
  Synthetic Construct
caggcgtgagccacca

SEQ ID NO: 60 - DNA, 30 bp, Artificial
  Synthetic Construct
aaagaaaattaagtctgactacactacagc

SEQ ID NO: 61 - DNA, 29 bp, Artificial
  Synthetic Construct
aggaccacaataggcaaaaaaaaaaaaaa

SEQ ID NO: 62 - DNA, 19 bp, Artificial
  Synthetic Construct
ggaccagccccaaatgtca

SEQ ID NO: 63 - DNA, 20 bp, Artificial
  Synthetic Construct
agatgacagaggctccatac

SEQ ID NO: 64 - DNA, 26 bp, Artificial
  Synthetic Construct
gctgtgagtaaaatccatcctaccta